(19) United States
(12) Patent Application Publication
(10) Pub. No.: US 2021/0402343 A1
(43) Pub. Date: Dec. 30, 2021

(54) DEVICES AND METHODS TO PREVENT TRANSFER OF INFECTIOUS AGENTS USING A MACHINE LEARNING MODEL

(71) Applicant: Thomas Danaher Harvey, Rockville, MD (US)

(72) Inventor: Thomas Danaher Harvey, Rockville, MD (US)

(21) Appl. No.: 17/395,590

(22) Filed: Aug. 6, 2021

Related U.S. Application Data
(60) Provisional application No. 63/069,230, filed on Aug. 24, 2020.

(51) Int. Cl. B01D 46/46; G16H 50/20; G06N 20/00; B01D 46/00

(52) U.S. Cl. CPC B01D 46/46; G16H 50/20; A62B 18/10; B01D 46/0028; G06N 20/00

(57) ABSTRACT

A system is described to protect a person from exposure to pathogens in breathing. A control device which can be an adjustable or removable filter, a valve to switch air sources, added substances interruption of the air stream is controlled by a determination made by a machine learning system with a machine learning model usually implemented as a neural network. The determination is made on the basis of training of the model which can be one or multiple stages with one stage in a large server not in real time and another stage in a field device which uses sensors and other information sources to acquire data to make a decision concerning the relative risk of various circumstances. Conditions detected include presence of other persons, level of activity of the protected person and many other factors concerning the surroundings, activity and history of the protected person.

### CROSS REFERENCE TO RELATED APPLICATIONS

This application claims the benefit of provisional patent application Ser. No. 63/069,230 filed Aug. 24, 2020 by the present inventor.

### STATEMENT REGARDING FEDERALLY SPONSORED RESEARCH OR DEVELOPMENT

Not Applicable

### BACKGROUND OF THE INVENTION

### Field of the Present Invention

The present invention relates to a method protecting persons from infectious diseases and allowing free breathing when risks are lowered.

### Background of the Current Invention

Certain diseases are transmitted by means of contaminated air exhaled by one person and inhaled by another, who is thereby exposed to the disease. The contaminated air may also be created by another source and may be specific to the vicinity of the person carrying the disease or may be in general circulation in an area.

This is currently addressed by having the person to be protected from exposure wear a mask to filter inhaled air. Effective filtration restricts the flow of air and is uncomfortable for the mask wearer. In addition, this discomfort can cause a person to forgo wearing the mask and thereby become exposed.

It is desirable to reduce the level of filtration at times of lower risk. Because the danger of exposure varies widely based on the presence of other persons, level of activity and many other factors. There are various ways to accomplish this based on a determination of the level of risk. Allowing the person wearing the mask to make this determination is unreliable and a better way is required.

The current invention addresses this problem.

### BRIEF SUMMARY OF THE CURRENT INVENTION

The current invention provides for free breathing by a Protected Person and for protection from the transmission of disease by controlling the transfer of air under the control of a machine learning system with a machine learning model. A mechanism is provided to allow free breathing and free transfer of air when the risk is deemed to be lower and to provide a restriction on the flow of air when the risk is deemed to be greater. The decision mechanism with called the Primary Control. The actual mechanism to restrict flow is called the Primary Valve.

The machine learning system is trained to recognize the level of risk based on information collected in real time and to control the airflow.

### BRIEF DESCRIPTION OF THE DRAWING

FIG. 1 is a diagram of the relationship between various elements of a system.

FIG. 2 depicts various systems which use sensors carried by the Protected Person.

FIG. 3 depicts various arrangements of a Primary Valve to control use of a mask

FIG. 4 depicts a meeting of two persons with one wearing a mask.

FIG. 5 depicts various sensors used to provide inputs to the Primary Control.

FIG. 6 is a diagram of the steps and structure of the basic information flow of a Machine Learning Model and its training.

FIG. 7 is a diagram of the process for developing a machine model.

### DETAILED DESCRIPTION OF THE INVENTION AND EMBODIMENTS

Definitions

The definitions given in this section are intended to apply throughout the specification and in the claims

Closed Condition

The term Closed Condition means a more restrictive condition to be accomplished by the Primary Valve.

Current Condition

The term Current Condition means that the Primary Control has determined that a more restrictive condition should be accomplished by the Primary Valve.

Open Condition

The term Open Condition means that the Primary Control has determined that a less restrictive condition should be accomplished by the Primary Valve.

Permeability of a Barrier

The term Permeability of a Barrier refers to the amount of a substance including air, a pathogen or another specific component of the incoming air to pass the barrier at a given pressure. It includes conditions such as the presence or absense of a filter and changes to the ability of the barrier to pass a substance based on control inputs such as presence of a second substance, electrostatic changes, movement of parts of the barrier or other changes to the barrier to control passage of substances.

Partial Condition

The term Partial Condition, which may also be called Partial Controlled Condition means that the Primary Control has determined that a particular state should be accomplished by the Primary Valve.

Primary Control

The term Primary Control is used to refer to the device or system which determines the desired state for the Primary Valve. It may combine or take into account rules, sensor readings or data from external sources. The determination may be further processed or

Primary Valve

The term Primary Valve is used to refer to the device which is controlled by an embodiment which allows relatively free flow in the open condition and restricted flow in the closed condition. It may be implemented by a physical barrier which is applied to a variable degree, by an air curtain or other flow of a fluid, or by change in the condition of a porous material which affects the ability of the controlled fluid to flow. It may either control air from a source which is to be excluded to prevent contagion or control an alternate source of air flow to replace or displace the flow which is potentially contagious.

Protected Person

The term Protected Person refers to a person wearing a mask which incorporates or uses a Primary Valve. Other persons may be protected by the use of the mask or there may be multiple persons with masks having Primary Valves operated by a Primary Control but the term Protected Person here refers to the mask wearer.

Description

Function of the Primary Control

The function of the Primary Control is to evaluate information to provide a Determined Condition.

Inputs to the Primary Control

The Primary Control can use information from a variety of sources. This information is assembled to make an assessment of the desired actions of the Primary Valve and produce a Determined Condition. Input can include data from sensors carried by a person—the Protected Person—wearing by the mask, data from sensors in the area or environment where the mask is being worn, reports received as data transmitted to the primary control, warnings transmitted by other persons and data from timers and schedules.

The information evaluated by the Primary Control is not limited to data collected by sensors. It can be used in conjunction with supplied data and variations in orders and rules from outside sources and authorities. Supplied data to the Primary Control can come from sensors in the environment. Examples would include sensors to detect persons with or without location data, sensors to detect wind velocity and direction and sensors to measure carbon dioxide from breathing. Other information may be transferred from other sources. It may concern conditions such as levels of infection in persons in the area or other defined sets of persons. It may also concern policies, analysis or conclusions from authorities or other persons.

Sensors carried by the Protected Person

Sensors carried by the Protected Person may include a camera which can produce images which can be analyzed for nearby persons, nearby data sources or environmental conditions. The camera can be built into a device such as a personal device (cellphone), or a special processing device to serve as a Primary Control, or it may be separate.

Temperature sensors when available can provide clues to the environment such as indoors or outdoors, state of the Protected Person, and presence of Air Conditions. All of these when processed by specific rules or with a machine learning model can help determine the level of need for mask protection.

Accelerometers and gyroscopes are common sensors that are of use in determining activities of a person carrying them. They may be available in a persons cellphone or in a special device used as a Primary Control. The movements of a person can provide clues to the persons state of exercise. For example, the Primary Control can open the Primary Valve when the person is actively running and close it at other times. A machine learning model can use movements detected by an accelerometers or gyroscope to determine if a person is sitting or standing. Both larger movements and small movements such as vibrations are useful in making such determinations. Properly trained machine learning models can make distinctions from data collected from actual or artificially generated examples. It is not necessary for any person to actually understand the meaning of the data or its analysis for this to work in typical uses.

Networking Primary Controls

Multiple Primary Controls can be networked. They can exchange raw sensor data, Condition Determinations, partially processed data and data received from outside sources and servers. Network connection can be managed by a single source or a protocol may be used to allow Primary Controls to join the network on an Ad Hoc basis. It may be desirable to control the data exchanged for privacy purposes. For example, data may be scrubbed of personal identification in conjunction with location. In such cases information as limited as identifying only the fact that a person is present in a certain area is transmitted. The information may be encrypted, so that only selected receiving systems or Primary Controls can acquire the information.

Processing in the Primary Control

The Primary control can do processing various strategies. Three classes of strategies are logical combination of inputs, transmission of information provided by other systems and using information from sensors, other systems and received transmissions as inputs to a machine learning model trained to generate a Determined Condition.

An example of a simple logical combination of inputs is using of strong signals of movement from an accelerometer with angular motions of a gyroscope to open the Primary Valve when a person is exercising and close it when the person stops exercising. Thus, a person could breathe freely when running but protect themselves and others when sitting with others after the run.

An example of using received information from other systems would be to receive signals send from a system to detect persons in a room. When two or more persons are detected the detection system built into the room would send a signal which would be picked up by Primary Control systems and used to close the Primary Valves of persons in the room.

An example of using a Primary control containing a Machine Learning system is to have a Machine Learning Model which is trained to distinguish patterns of movement based on inputs originating in an accelerometer and gyroscope. Those patterns indicating a likelihood of not having persons nearby would produce a different Desired Condition than those indicating a likelihood of nearby persons.

The Primary Control can also produce information and outputs other than the Determined Condition for the additional control of the Primary Valve and other actuators.

Machine Learning Model for the Primary Control

Many implementations will use a machine learning model to evaluate information collected to produce a Determined Condition. The machine learning model works by being trained with collected data and then evaluating collected data. The steps are shown in Fig's 10 and 11.

Function of the Primary Valve

The function of the Primary Valve is to allow easier breathing for the protected person in situations determined to have less risk and to provide better protection in situations with more risk.

Situations Where Control is Useful

Situations where control of airflow in a mask is useful include those where the wearer is actually or potentially engaged in temporarily increased activity. This would include situations such as running or vigorous walking. In that case not only is there a need for free breathing but the motion itself can break contact with other persons and reduce the need to filter air. Similarly engagement in a specific activity is often correlated with reduced need for a Closed Condition of the Primary Valve. An example would be a manufacturing work station with its own protection against hazards or local ventilation.

Detailed Description of the Drawing and Certain Embodiments

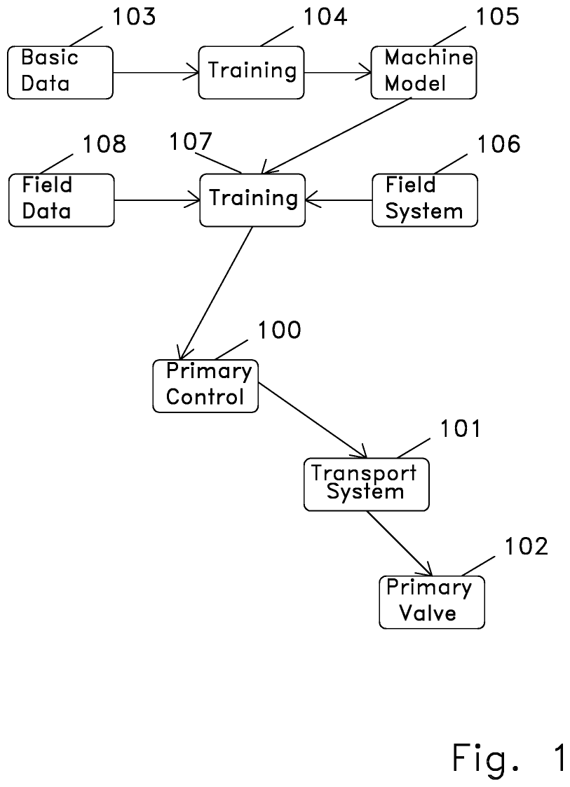

Referring to FIG. 1.

A diagram of the relationship between various elements of a system is depicted. The Primary Control 100 makes a determination of the desired Condition and uses the Transport 101 system to control the state of the Primary Valve 102.

Basic data 103 is collected or downloaded from a preexisting collection and used to (pre) train 104 a machine learning model 105. The model is then downloaded to a field system 106 and further trained 107 with data collected in the field 108 or from the particular environment or subjects to be evaluated in the use of the model.

The pre-trained and trained model is further downloaded to the Primary Control. The Primary Control evaluates the mode with inputs from sensors and data from external systems and databases and generates commands for a Desired Condition which are transported to the Primary Valve for execution and establishment of the Desired Condition.

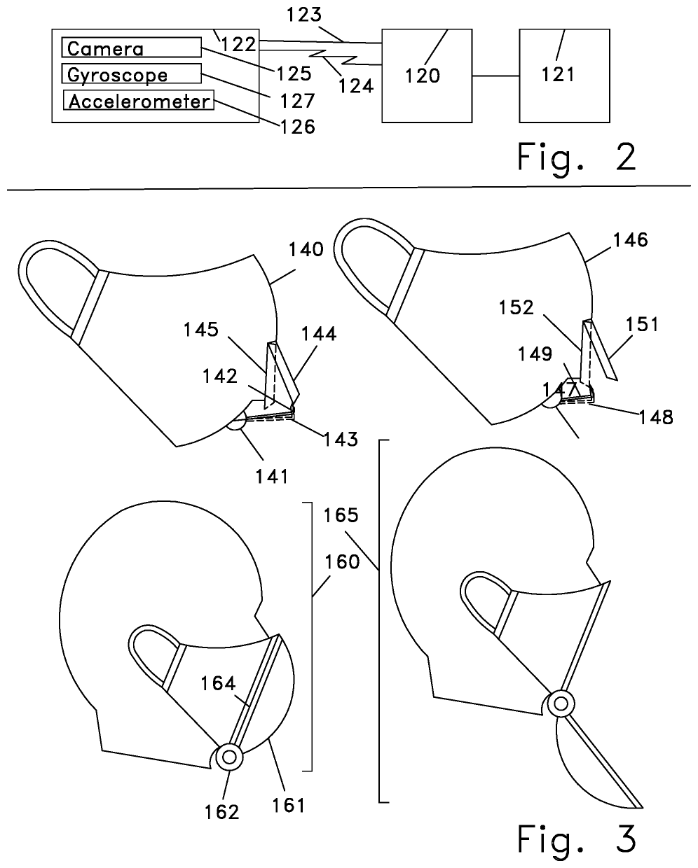

Referring to FIG. 2.

FIG. 2 depicts various systems which use sensors carried by the Protected Person 122 as inputs to the Primary Control 120 to operate the Primary Valve 121. The Primary Control may be connected to the Primary Valve by hard wiring 123 or by a wireless connection 124. A camera 125 worn by the Protected Person provides data analyzed by a machine vision system detects persons nearby and the Primary Valve is operated in a Closed Condition. In more complex cases information from motion sensors such an accelerometer 126 or gyroscope 127 is combined with the camera information to provide inform the evaluation.

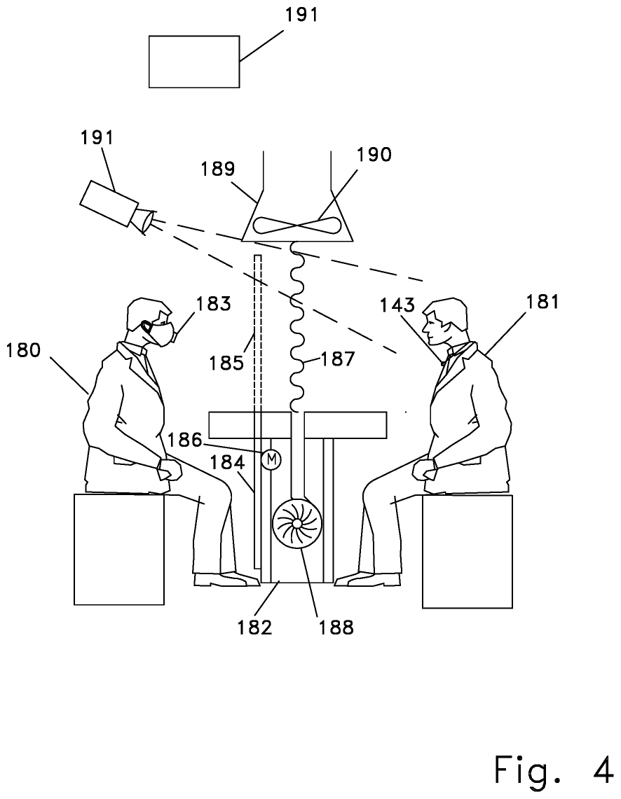

Referring to FIG. 3.

FIG. 3 depicts various arrangements of a Primary Valve to control use of a mask 140 to provide controlled protection. In a first configuration with the Open Position producing free bypass of the mask on both Inhale and Exhale, an actuator can 141 move a pawl 142 between the positions for the Open Condition 142 and the position for the Closed Condition143. With the pawl in the Open Condition a flap 144 is caught when moved outward by an exhaled breath and the valve remains open until the pawl is retracted by the actuator. If the pawl is retracted to position 143 then the flap closes 145 on inhaled breaths and air is filtered through the mask in one direction only.

In a second configuration of a mask 146, the Open Position produces free bypass on exhale and mask filtration on inhaled breaths. The Closed position produces filtration on both inhaled and exhaled breaths. An actuator can 147 move a pawl 148 between the positions for the Open Condition 148 and the position for the Closed Condition149. In the Open Condition the flap 150 is free to moved from an open position151 with free exhale breathing when pushed outward by an exhaled breath to a closed position 152 which requires air passage through the mask with filtration.

In a third configuration a front panel 161 of a mask 160 is hinged at a rotary actuator 162. In the Closed configuration 163 the front panel is sealed at a joint 164 to the mask. In the Open configuration 165 the rotary actuator pivots the front panel to all free breathing with the mask out of the way. There is a side benefit of visibility of the mouth and ease of speech to allow better oral communication especially with persons who require at least partial lip reading.

Referring to FIG. 4.

FIG. 4 depicts a meeting of two persons with one wearing a mask with a Primary Valve controlled by a Primary Control and an additional Primary Valve built into the counter where the two meet. A first 180 and second 181 person are seated at a counter 182. In the depicted scenario the first person is being protected from possible infection by the second person.

There are three subsystems that function as Primary Valves depicted. The first 183 is a flap or other flow controlling device in a protected person's mask. The second is a shield 184 which normally retracted but which can be quickly raised into a deployed position 185 by a motor 186. The third Primary Valve is an air curtain 187 which is created by a blower 188 mounted in the counter. The air curtain is captured by an overhead duct 189. This duct is also used in some scenarios to exhaust air and provide a lessor level of protection with the aid of a fan 190. In other scenarios the Primary Control can turn on fan or blower 188 or 190 to provide air which takes the place of air excluded by the Primary Valve. In the case the fan or blower becomes an additional or Secondary Primary Valve.

The Primary Valves are operated by separately or in combinations by a Primary Control 191. The primary control uses information with a processor which may use a machine learning model with additional training to the model from data acquired from the persons protected. It may have training done prior to installation which is used with further training from data acquired from persons using the installation prior to the particular persons shown protected here.

The Primary Control uses data collected by sensors several of which are shown as inputs to evaluation using the trained machine learning model to determine when to apply one or more varieties of a Closed Condition and to actuate one or more of the Primary Valves. One sensor is a camera 191 observing the second person. It looks for actions which indicate a need for a change of condition. For example, a machine learning model can be trained to detect when a person is likely to sneeze or cough in the next short period. This can be done from camera or visual data, or it can be done from audio data connected by a lavalier microphone 192 worn by the second person. The Primary control can also take into account other information such a elapsed time. Other sensors could measure air currents, or shifts of weight on the seats.

The Primary Valve subsystems are adapted to provide efficient communication between the persons in an Open Condition and effective protection in a Closed Condition. For example, the air curtain 187 could be quite noisy when in operation but very quiet in other conditions such a in a ready only condition. It is not limited to two states but can be partially operative for some protection with reduced noise. The shield 184 or 185 would typically have safety provisions to allow rapid deployment with safety protection for persons who might be in its way.

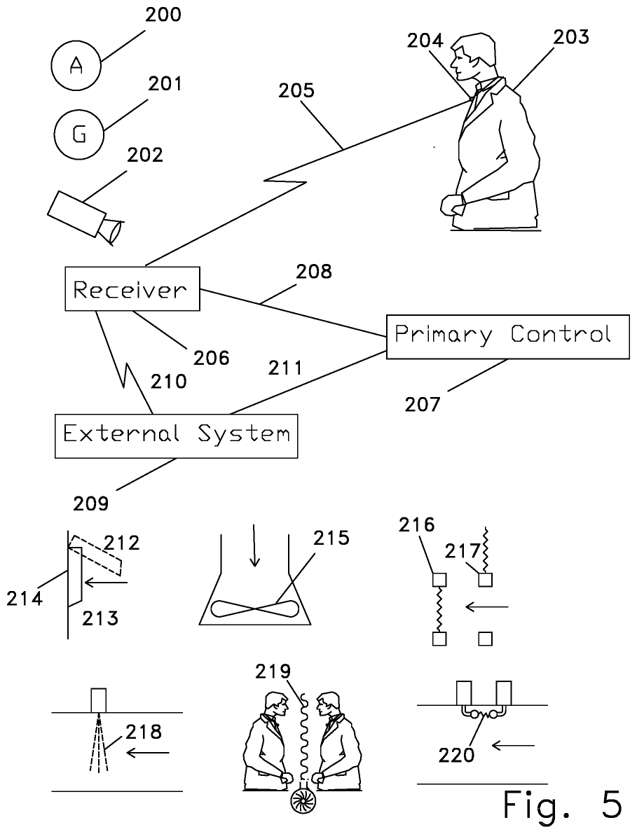

Referring to FIG. 5.

FIG. 5 depicts various sensors used to provide inputs to the Primary Control, the Primary Control and various Primary Valves. An Accelerometer 200 can be used to detect activities of the Protected Person or other persons. The level of activity may be a major factor in the estimation of danger of transmission of infection. A person with a high activity level may be breathing heavily with more air flow or may be distracted from taking other care to prevent infection. A gyroscope 201 alone or in combination can perform a similar function. A camera 202 can detect persons in the area and other conditions that affect the determination of potential transmission. Many other sensors are available which can detect specific conditions such as the breath rate of the protected person.

A person 203 wearing a transmitter 204 can send a signal 205 to a receiver 206 notifying the a Primary Control 207 connected 208 to the receiver of the presence of the person. Data collected from sensors, databases or other sources can be analyzed by an external system 209 and transmitted to the receiver 210 or directly 211 to the Primary control.

After data is evaluated by the Primary Control control instructions is passed to devices to implement the changes in required air access or airflow. Methods of implementing this access include opening 212 and closing 213 a solid barrier 214 including the gate in a valve, starting and stopping a fan or blower 215, moving a filter into 216 or out 217 of the air path, injecting substances 218 such as disinfectants or warning odorants into the air flow, controlling an air curtain 219, controlling an electrical discharge 220 which may kill organisms or produce ozone for warning smell or purification.

### Figures Concerning Machine Models

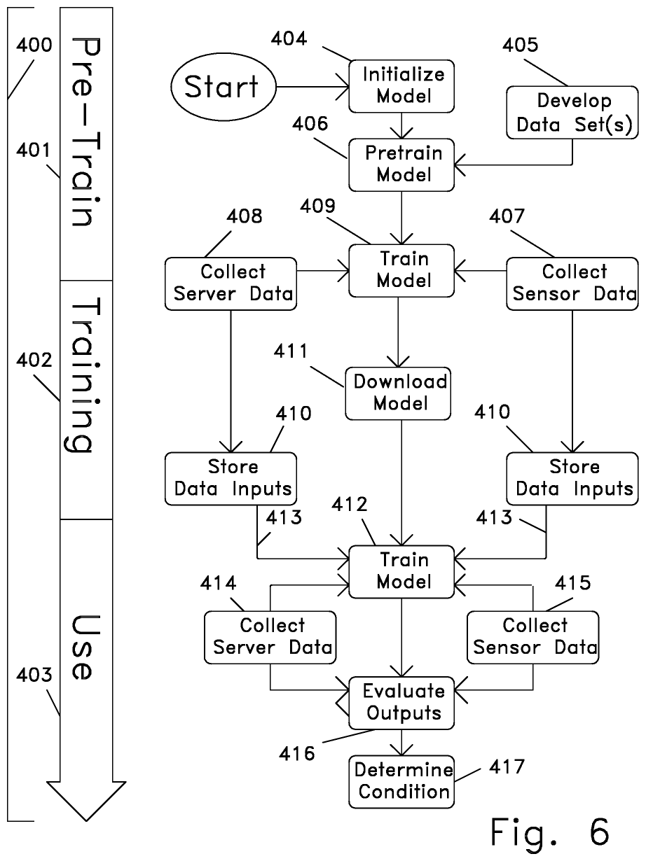

Referring to FIG. 6.

There is a timeline 400 which is divided into three periods. This represents phases of the data use in developing and using a machine model to perform necessary functions operating a Primary Control.

Pre-Training 401 is a phase used to develop the functions of a Machine Learning Model which are not dependent of the design details of the application for a Primary Control. It develops abilities for the model to perform tasks such as recognizing people from sensor images or data. Special data sets are developed 405 and used to Pre-Train 406 an initialized 404 machine learning.

In addition, to the training on tasks which are applicable to a wide range of applications, specific recognitions may be trained in Pre-Training which are used in the specific intended use. These may be specific to the application that a second processor for evaluation of the model is implemented, but they would not depend of gathering data from the specific field case to be analyzed in the use of the machine model.

Training 402 is a phase used to train the model to perform specific data analysis relevant to the application of a Primary Control. The data is relevant to this task and may include sets such as sensor data for situations with and without other persons present. Data collected from sensors 407 and servers 408 which are in situations or simulated situations which affect a Condition Determination are used for the training 409. Data input collected in this phase may be stored 410 for use as an additional input in the later phase to provide context. In the drawing this training level is shown on the line between Pre-Training and Training because it can use data for generic applications and specific data collected for the particular application. Data for the specific application would here be collected in advance of the real time use of the system and so is distinct from the data collected in real time shown in 414 and 415.

The use or field phase 403 may or may not include additional training of the model with data collected while the Model is being used for evaluating inputs and producing Condition Determinations. This training 412 can occur after the Machine Learning Model has been downloaded 411 to a different processor for field use. Field data is collected and used for this training. It may be combined 413 with stored 410 data from the earlier phases to provide a context or for preliminary processing to provide suitable training inputs for the Machine Learning Model.

An example illustrating the usefulness of training in steps would be recognition of situations with new people as opposed the people who already in contact with the Protected Person. The Pre-Training can enable a Machine Learning System to do difficult tasks such as recognizing particular persons. They could then be checked to predict their contribution to the dangerousness of a particular situation. Much less processing power and time is needed to train a Machine Learning System on the particular situations encountered after it is moved to a real time field device. It could then be trained to recognize different patterns of movement and interaction which would correlate with dangerousness without the need to recognize particular persons.

Other simple recognitions easily trained include recognition of the Protected Persons resting movements or breathing, Recognition of the normal sound patterns in the location, recognition of the timing and scheduling of normal events in the field situation and recognition of breathing in a well fitted mask. These and many more situations are correlated to the danger level at a particular time and can be used to control a Primary Valve. They require training of the Machine Learning Model in the field situation after download of the model, but do not require extended processing on large processors in the manor of the first level of training.

The trained Machine Learning Model is then ready to evaluate 416 field inputs collected from sensors 415 and delivered from servers 414 to make evaluations of the model which are used, possibly with further processing of a different type, to generate 417 a Determined Condition.

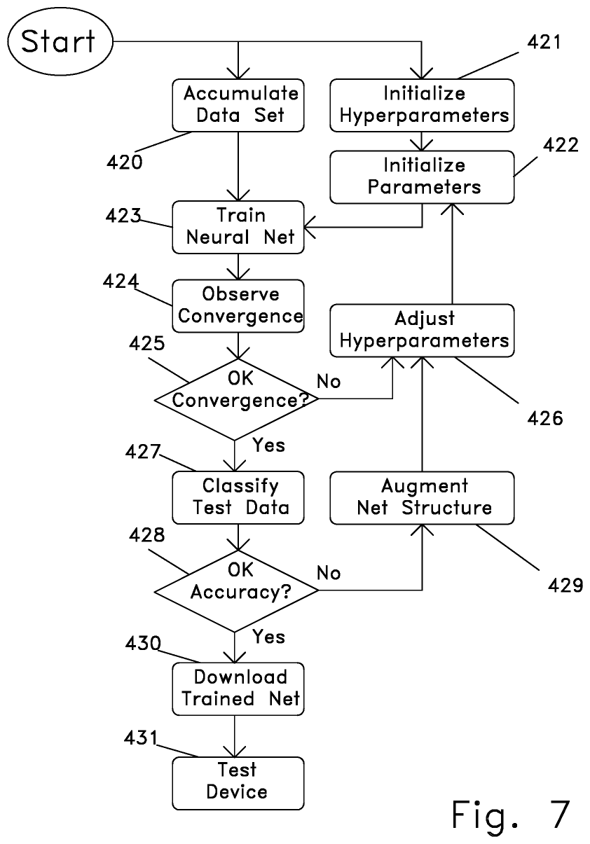

Referring to FIG. 7:

A diagram of the process for developing the pattern matching software for the system of FIG. 6 is shown. The software to be developed is a recurrent neural net with an input layer, a recurrent layer, a additional fully connected hidden layer and an output layer. Other more complex structures are also easily implemented but this structure would be sufficient for the low level of accuracy needed in many embodiments. This process can be repeated at different levels such as shown at 401 and 402 of FIG. 6.

The first step of the development is to accumulate 420 a data set for training and testing. The data set is typically divided and one portion used for training and another for testing accuracy. It is important that the portions used for these two steps do not overlap.

The data is divided into two sets with a larger portion for training and a smaller portion for accuracy testing. This is considered labeled data if it contains both input (sensor) data and the desired output for that data. Training with labeled data is easier and faster, but there are effective methods to train with unlabeled data.

In this and related embodiments, a step in the development which might be started in parallel with data collection is the design of an appropriate neural network. The sizing of the layers and the setting of various factors in the neural net which are in addition to the factors and values (parameters) that are adjusted in training are collectively referred to as hyperparameters to distinguish them from the “parameters” which are adjusted in training the neural network. The hyperparameters are initialized 421 to appropriate values. In some systems that are taught hyperparameters are adjusted during the course of training but are distinct from trainable parameters because the adjustments are on the basis of the progress of the training rather than being direct functions of the data.

The next step is to initialize 422 the parameters which are to be trained. Appropriate initialization is necessary for reasonably rapid convergence of the neural net. A number of techniques are taught to product an initial set of values which produced good training progress.

The network is then trained 423 by passing data set items through the network as implemented on a training processor. Because training requires larger processing power and time than use of the network after training special powerful processors are used for this step. The training process adjusts the parameters incrementally on the basis of the output of the neural network. The hyperparameters specify the methods of calculating the adjustment to parameters. Generally, the output of the network is used to back propagate through the network to provide further input to the adjustments. The items in the training portion of the dataset are used repeatedly while the convergence of the network is observed 424 by processes in the training data processor.

If the convergence is judged 425 not to be adequate the training is stopped, the hyperparameters are adjusted 426, the neural network is reinitialized and the training process is repeated until satisfactory convergence is obtained. The smaller portion of the data set which has been retained and not used for training is then passed 427 through the neural network (classified) and the output is checked 428 for accuracy. If accuracy is not sufficient for the goals of the particular system being developed then the net structure is made larger 129 and the training process is repeated until satisfactory accuracy is obtained.

The trained neural network is then downloaded 430 to the target device, which is then ready for system testing 431.

Description of Machine Learning, Models and Training

Processor, Machine Learning and Models

A device is described to use gathered information to solve complex problems in interpreting incoming data and generating a plan for escape. In general, it is not feasible to discover all of the rules and relationships necessary to solve that problem and to write a determinative computer program that produces a sufficient result. However, methods have been developed and are wide and increasing to use a set of examples which is processed and used to product a set of rules which working together can find answers suggested by the examples. A substantial set of examples and a large amount of processing are required but many people are trained and are being trained in application of well-known methods to implement this approach on a wide variety of problems.

There are limitations on the kind of problems that can be solved with this approach, but the problem here is well suited for the approach because of the specific input data that is to be gathered and because of the specific kind of output that is required.

Machine learning as used here and in the Claims is a term for the type of artificial intelligence that is implemented without explicit programming to generate solutions to the problems confronted. It is focused on the development of solutions by having the effective program change as it incorporates data in such a way that it converges on an ability to produce the required solution.

Model

The term model as used in this specification includes representations that do not explicitly describe the system modeled but which are designed or trained to produce information based on the behavior of the system. It includes pattern matching system such as a neural network which has been trained on data exemplifying the system. In that case the model consists of an (possibly huge), array of parameters that determine the operation of the individual neurons in the neural net program. Training would work by systematically adjusting the values of these parameters on the basis of the training data.

Machine Learning

Machine learning is a well-developed and understood method of creating devices that are capable of solving problems that defy development of programmatic solutions that rely on understanding in detail the working of the system to be analyzed. A famous example is the modern language translation systems widely used on personal computing devices. Development of programs to translate languages has produced poor results because of the complex and subtle structure of human languages and the scale of the problem. But systems have been developed to be trained on a large (possibly hundreds of millions) number of examples of language usage. The trained models are then applied to an input in one language and provide output which is very likely to be a satisfactory translation in another language of that input.

Machine learning systems are very different from computers running programs written to model problems to be solved. While the implementation of a machine learning system may be made by means of a computer program, this is not the only way to implement machine learning models. An array of analog devices (usually called gates) can implement the model in a massively parallel way. Rather than containing a program, a machine learning system constructs a model which transforms an input through a huge number of gates to produce an output which has a statistical meaning. The operation of the gates is modified in the training steps until the behavior of the model converges on a tendency to produce desired results.

Machine Learning System Models

A machine learning system model or just “model” as used in this specification and in the claims is a large set of parameters represented as data or physically and arranged in such a way that they can be adjusted by a training process based on a collection of data that represents the system being modeled. The model also allows inputs that represent a particular state or set of states of the system to be analyzed by use of the model. The use of the model transforms the inputs into a set of outputs that constitute an analysis of the states being analyzed.

A model can be applied to a set inputs by means of an algorithm executed by a processor or by means of a physical analog device to perform the transformation. The algorithm or device is only the means of evaluation and is distinct from the model which is the set of trained parameters and the structure in which they interact.

Training a Model

In this specification and in the claims the process of training a model consists of applying data representing possible inputs to the machine learning system with the model in its current state of possibly partial training. The outputs of the system are used to generate incremental adjustments to improve the transformation of the inputs into outputs better representing the desired behavior of the system.

The usual way to determine the adjustment to be made to the model for each group of inputs presented is to calculate or measure the effect on the outputs of each parameter in application of that set of inputs. If the effect is favorable in providing outputs that correspond as wanted to the inputs, then the parameter is very slightly augmented to improve the overall behavior of the model as trained.

There are many ways to accumulate the data sets used for training. One way is to find or set up a large number of examples with known outcomes and collect the data from them. Another way is to write an algorithm which generates examples. The examples can be graded by people or the generation method may be able to predict the outcomes. Some problems are easy to solve in reverse; i.e. a set of inputs may be easier to get from a assumed output than to find the output from a set of inputs. For example, to train a system to distinguish pictures of dogs from pictures of cats one can get pictures from public sources such as the internet and use humans to label the species depicted. That set can be used to train a model which can test other pictures.

Convergence

The training process is continued for each item in the training set data. Because it is important that training result in a stable and gradual progression of the model toward the desired behavior teach round which uses the set of training data items only changes the model by a small increment. The rounds are repeated many times and the results are compared to data reserved for testing in order to measure the effectiveness of training. If the structure of the model is well chosen than parameters will converge on values that produce the desired outputs for various input sets.

Training in Levels

Models can be arranged in levels both for training and for evaluation of inputs. The application of the model to a set of inputs generates outputs that describe in a higher level of generality the meaning of the inputs. Those outputs can become inputs to further structure which is a model for a more general transformation of the original inputs toward meaningful outputs.

In this specification and in the claims, a level of training is the training of a portion of the parameters of a model to produce outputs that are trained until a state of convergence is attained and made available for input the next portion of the model. That is, distinct levels are made distinct by separate training to convergence. It is possible to simultaneously train multiple levels, but they are distinct levels when they are separately tested for convergence. A level that is not tested for convergence but which uses inputs from a level that has been brought to convergence is a distinct level from the level providing the inputs.

Typical models are in at least four levels. The first which here is called the Basic level takes raw sensor input and describes it in terms directly definable based on the input data. Examples would be detection of edges from visual data and of tones, harmonics and burst timings for audible data. The second level which is here called the General level is to identify objects and events from the output of the first level. Examples would be to detect a person crossing the path of the sensor or identifying a sound as a gunshot or crowd noise. The third level, herein called the Specific level is to allow the model to identify actions and objects appropriate to the purpose of use of the model. Examples of this level include model layers to implement steering or acceleration of a vehicle or determination of compliance with a standard in a specific type of situation. There is also a fourth level called the In-Use level in many implementations. This level incorporates data collected while a model is in use which modifies the model to allow evaluations at a later time to take into account earlier inputs or evaluations where a series of evaluations is made.

Implementation of Training on a Processor with a Memory

Training requires a very large amount of processing to apply the large amount of data in the training set repeatedly to incrementally cause the model to converge on the desired behavior. If the adjustments from one pass through the data are too large, then the model may not converge or may not allow the effects of all of the inputs to diffuse through the model structure and correctly operate. For this reason, specialized very powerful processors are used for training. They are not appropriate for incorporation in portable devices because of considerations of size and expense.

Basic Training

In this specification and in the claims, basic training refers to training which is used to interpret inputs from sensors or raw data from data sources to identify aspects of objects and actions treated as objects that are implied by the data and too general in nature to identify the potentially detected objects at this stage. Examples include edge detection, categorization of sounds by location of the source, face detection, orientation in space, counting objects, elimination of backgrounds and many other general tasks of interpretation.

A portion of a machine learning model with this training can be used for many applications and could be supplied by a specialized developer. It's training would be brought to convergence and the outputs supplied to the next level of training when the model is used to evaluate inputs either for further training of other levels or in actual use.

Data for General Training Describing the Area of Application of a Model

Data for the general level of training can be acquired by collecting a number of real examples or by generating examples with a program to produce examples and training data. In this and in other levels, it is often much easier to produce a program for generating examples for machine learning than to determine specific rules to allow determinative non-learning algorithms for evaluating rules designed for human understanding.

Transferring a Trained Model

Levels of training of a machine learning model can be divided into two classes. The first class is those levels that require very large amounts of processing power and time to train. These typically use large training sets and are done before other levels. They include in most cases the basic training levels which are concerned into extracting interesting features from raw data usually from sensors and the general training levels which concern coordination of features in sets of circumstances which are designed to encompass the specific situation to be evaluated. These levels cannot be conveniently handled in real time and on a processor convenient to take into the field to perform evaluations.

The second class of levels are those that must be performed after the specific situation to be evaluated is determined. They must be performed in real time and on processors available at that time. The model trained by the first class of levels can be transferred to a more convenient processor for the second class of levels of training.

A typical way to use this structure of two classes of training is to create a model which is trained for recognition tasks on large servers over a period of time. This model may be quite expensive in development but can be used for multiple applications. The model is then extended with additional layers which are trained in tasks needed for the particular application. At this point the model would be tested and downloaded to the device which will apply the model in real time. Additional layers are added to the model to handle tasks which can benefit from simpler training involving data collected in the field by the application device. This additional training because of time and processing power constraints is limited in intensity but is still useful because of it's involving more specifically applicable data.

Data for Testing in Particular Applications

After a model is trained in several levels and downloaded to a processor to use the model to evaluate situations, data must be collected with an appropriate sensor. The data is provided to the processer as input to the model for an evaluation to produce outputs. The outputs may have further non-machine learning processing to produce a determination from the model in use.

Acquisition of Testing Data with a Sensor

A portion of the data collected or generated at each level is reserved for testing. This data is not used for training to enable testing the model without concern that the model might be effect only with the specific cases used for training.

A Trained Model as a Special Purpose Machine

Once a model is trained and put into an environment that allows it to evaluate sets of input data the combination becomes a special purpose machine for making the determinations for which the model was trained. The feedback that is used to adjust parameters to produce desired outputs from inputs has created a network that can operate on other inputs to produce similar results. This behavior has been tested and the machine can be put into use.

I claim:

1. A device to prevent transmission of a pathogen to a person wherein:
air for breathing by the person is provided in a multiplicity of states of the device including:
(a) a first state where the air for breathing by the person is provided to the person with greater ease of breathing to than in the second state, and
(b) a second state where exposure of the person to the pathogen from the air for breathing by the person is reduced from exposure of the first state; and wherein:
a machine learning system with a machine learning model makes a determination of the probability of transmission of the pathogen on the basis of the determination changes the state of the device and the device is placed in a specific state selected from the first state and the second state on the basis of the determination.

2. The device of claim 1 wherein:
the second state passed the air for breathing through a filter and the first state bypasses the filter.

3. The device of claim 2 wherein:
the machine learning model is trained on a first processor,
the machine learning model is subsequently downloaded to a second processor,
the machine learning model is trained on the second processor with data from the sensor gathered subsequent to the downloading of the machine learning model to the second processor, and
the machine learning model is used subsequent to the training on the second processor to make the determination.

4. The device of claim 1 wherein:
the second state passed the air for breathing to be supplied and the first state stops the supply of air for breathing.

5. The device of claim 4 wherein:
the machine learning model is trained on a first processor,
the machine learning model is subsequently downloaded to a second processor,
the machine learning model is trained on the second processor with data from the sensor gathered subsequent to the downloading of the machine learning model to the second processor, and
the machine learning model is used subsequent to the training on the second processor to make the determination.

6. The device of claim 1 wherein:
The determination is made on the basis of presence of a second person.

7. A device to prevent transmission of a pathogen from a first person to a second person wherein:
a machine learning system makes a determination of the probability of transmission of the pathogen by means a first air flow from the first person to the second person; and
on the basis of the determination at least one of:
(a) the permeability of a barrier to the flow of air is reduced to reduce the probability of transmission of the pathogen from the first person to the second person,
(b) air is supplied from a source producing a second air flow,
(c) air in the first flow is disinfected, and
(d) the first air flow is interrupted.

8. The device of claim 7 wherein:
the machine learning model is trained on a first processor,
the machine learning model is subsequently downloaded to a second processor,
the machine learning model is trained on the second processor with data from the sensor gathered subsequent to the downloading of the machine learning model to the second processor, and
the machine learning model is used subsequent to the training on the second processor to make the determination.

9. The device of claim 7 wherein:
the permeability of a barrier to the flow of air is reduced to reduce the probability of transmission of the pathogen from the first person to the second person,

10. The device of claim 8 wherein:
the permeability of a barrier to the flow of air is reduced to reduce the probability of transmission of the pathogen from the first person to the second person,

11. The device of claim 7 wherein:
air is supplied from a source producing a second air flow.

12. The device of claim 8 wherein:
air is supplied from a source producing a second air flow.

13. The device of claim 7 wherein:
the first air flow is interrupted.

14. The device of claim 8 wherein:
the first air flow is interrupted.

15. A device to reduce the risk of a person to exposure to a disease agent wherein;
a person breathes air from which may contain the disease agent,
the air from the source is modified in at least one of its availablity, flow, source, or contents to reduce the risk of exposure to the disease agent on the basis of a determination by a machine learning system employing a machine learning model.

16. The device of claim 15 wherein:
the determination is made on the basis of data from a sensor gathered while the person is breathing the air.

17. The device of claim 16 wherein:
the machine learning model is trained on a first processor, the machine learning model is subsequently downloaded to a second processor, the machine learning model is trained on the second processor with data from the sensor gathered subsequent to the downloading of the machine learning model to the second processor, and the machine learning model is used subsequent to the training on the second processor to make the determination.

18. The device of claim 17 wherein:
the modification of the air from the source is made by selecting one of passing the air through a filter and bypassing the filter.

19. The device of claim 17 wherein
the modification of the of the air from the source comprises:
selection of the source of the air.

20. The device of claim 17 wherein
the determination is made on the basis detection of the presence of a second person.
